(19) United States
(12) Patent Application Publication
(10) Pub. No.: US 2014/0296630 A1
(43) Pub. Date: Oct. 2, 2014

(54) Medical Osteotome Assembly

(71) Applicant: Paul A. Zwirkoski, Pinckney, MI (US)

(72) Inventor: Paul A. Zwirkoski, Pinckney, MI (US)

(21) Appl. No.: 14/241,802

(22) PCT Filed: Aug. 31, 2012
(86) PCT No.: PCT/US2012/053253

§ 371 Date: Mar. 19, 2014

Related U.S. Application Data
(60) Provisional application No. 61/575,845, filed on Aug. 30, 2011.

(51) Int. Cl. A61B 17/16; A61B 1/055; A61B 17/00

(52) U.S. Cl. CPC A61B 17/16; A61B 17/00234; A61B 1/055
USPC 600/104; 606/84; 600/109

(57) ABSTRACT

Described is a medical assembly and delivery devices for performing minimally invasive medical procedures. In particular, described is a controllable articulating directional tip assembly consisting of interlocking segments creating an articulating directional tip and various components utilized with the directional tip for various tissue alterations and/or viewing within a tissue cavity such as, for example, non-soft tissue alterations such as bone within a non-soft tissue cavity. In particular, the disclosed device could be utilized as an osteotome device for altering, such as cutting, bone.

### FIELD

Described herein is a medical assembly and delivery devices for performing minimally invasive medical procedures. In particular, described is a controllable articulating directional tip assembly consisting of interlocking segments creating an articulating directional tip and various components utilized with the directional tip for various implant delivery, tissue alterations and/or viewing of tissue within a tissue cavity such as, for example, alterations of non-soft tissue such as bone within a non-soft tissue cavity. In particular, the disclosed device could be utilized as an osteotome device for altering tissue, such as cutting or crushing bone.

### BACKGROUND

Currently, there are a multitude of various directional tip medical devices available for use in surgical procedures on mammals. Many of them are utilized to position certain devices such as endoscopes, orthoscopes, probes, catheters, and similar devices in specific locations within a body tissue. Procedures include tissue repair, tissue cutting, tissue removal or imaging. Most of these devices, however, are flimsy and lack the robustness and strength to be utilized for the objectives of altering, preparing and/or removing tissue, and in particular, non-soft tissue such as bone tissue. There exists a need for an articulating directional tip devices and assemblies that can be precisely placed within a tissue cavity, and in particular, a non-soft tissue cavity, for altering non-soft tissue such as bone tissue to accomplish desired objectives such as tissue alteration, preparation, cutting, compacting, removal, suctioning, and viewing, and devices and assemblies that have sufficient strength to accomplish the desired goal.

### SUMMARY OF INVENTION

Described herein is an articulating directional tip assembly consisting of interlocking segments that are connected by a flexible linking guidance wire and can be articulated through the use of a tensioning wire component that can cause the articulating directional tip be precisely manipulated within a tissue cavity while delivered through minimally invasive surgical procedure. In an alternative embodiment, the articulating

[symbol]

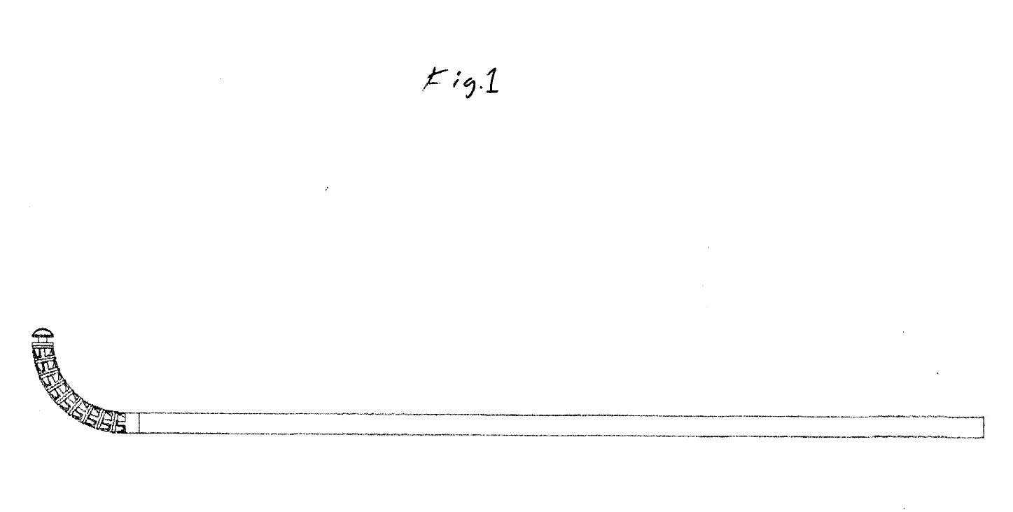

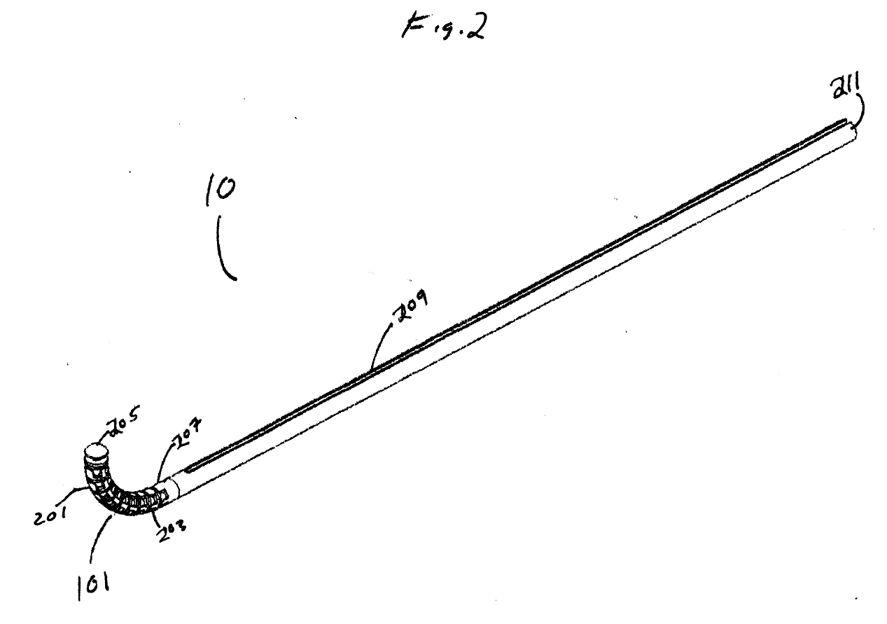

FIG. 1 shows a preferred embodiment of an articulating directional tip assembly with a directional articulating tip device.

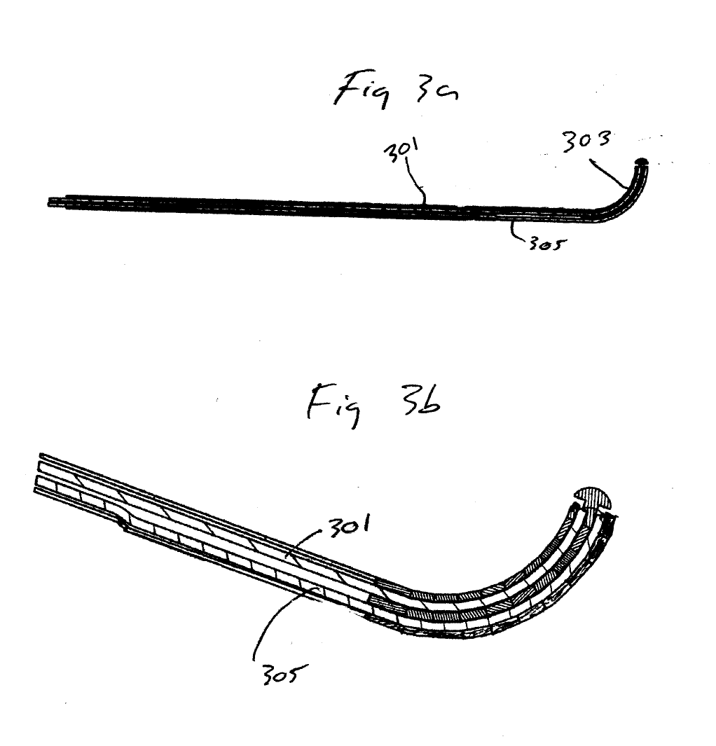

FIG. 2 shows a side view of an articulating directional tip assembly.

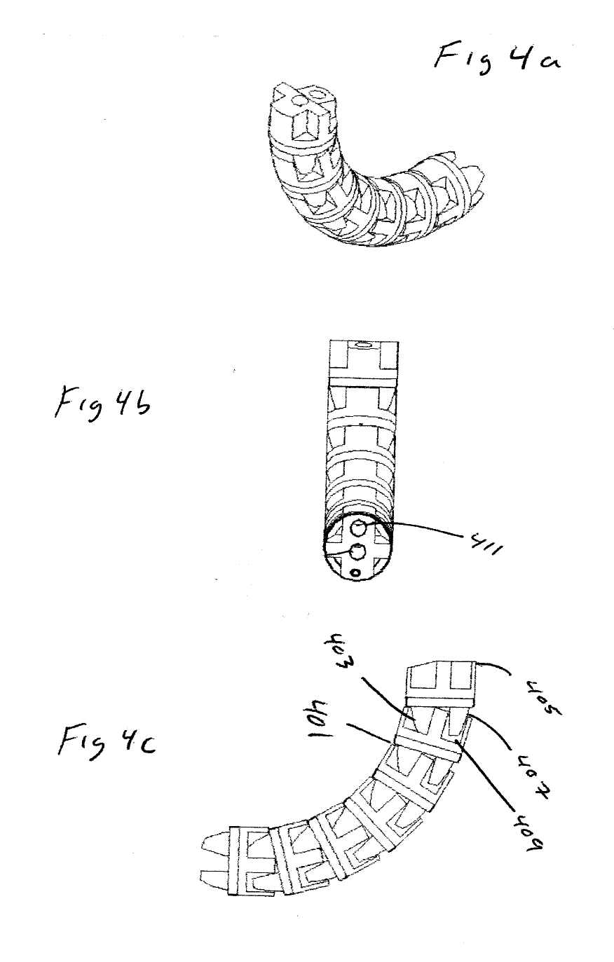

FIG. 3a shows a cutaway view of an articulating directional tip assembly with a flexible linking guidance wire and a tensioning wire component.

FIG. 3b shows a close up view of a cutaway of an articulating directional tip assembly.

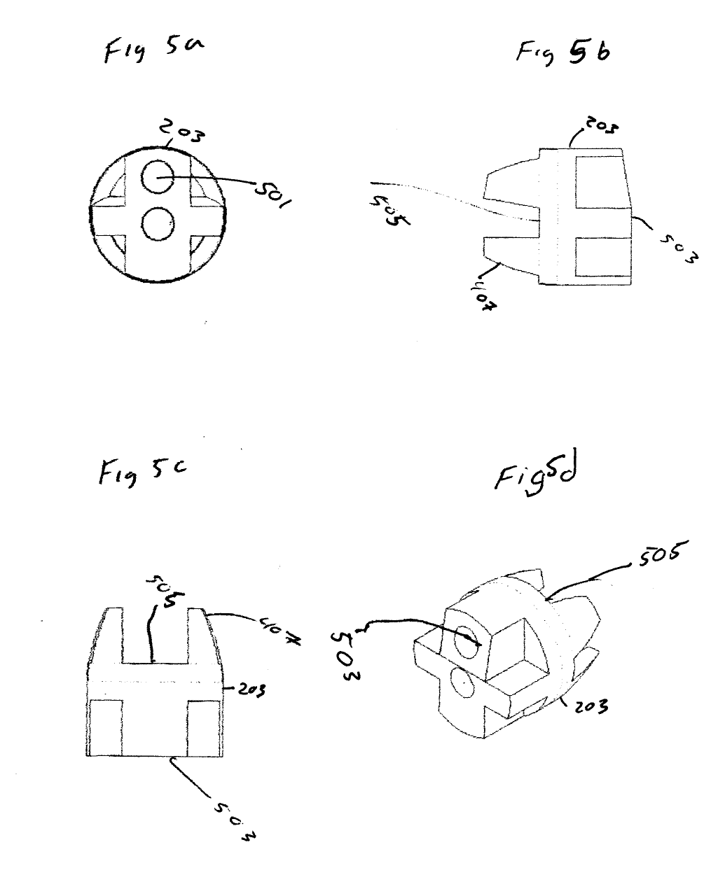

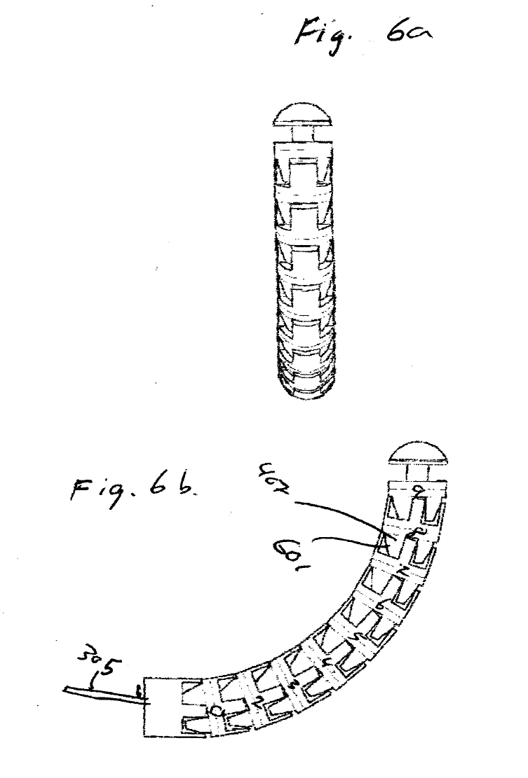

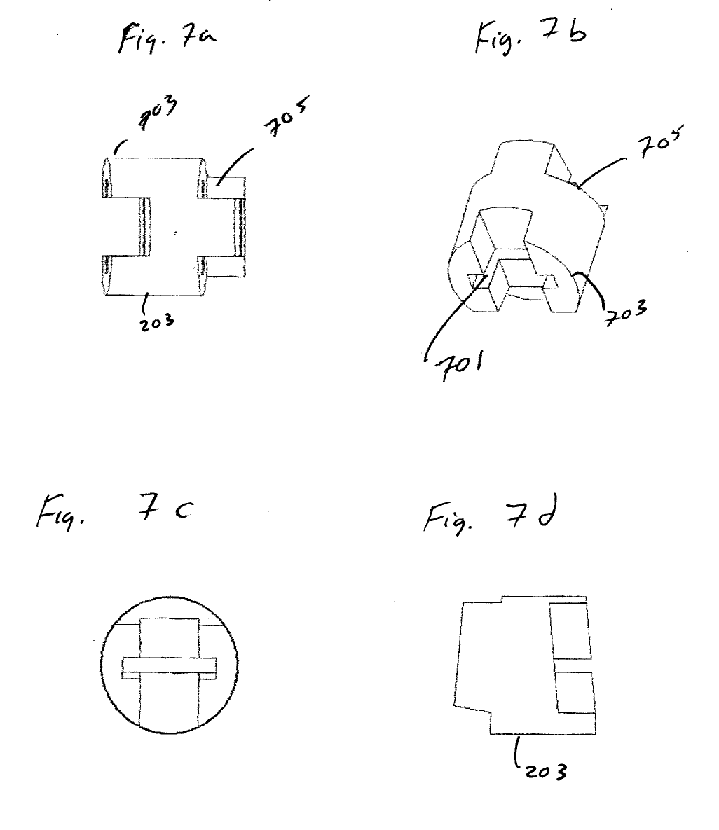

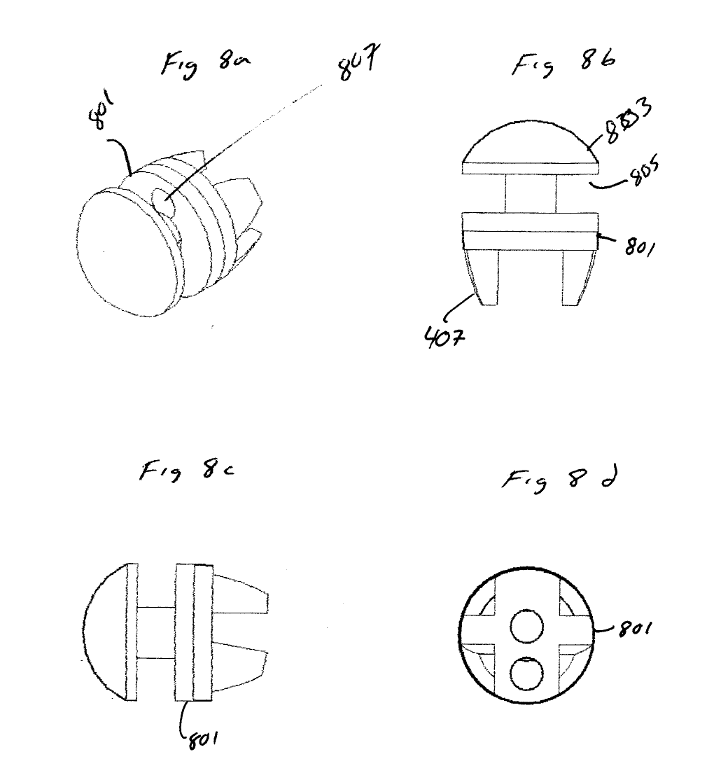

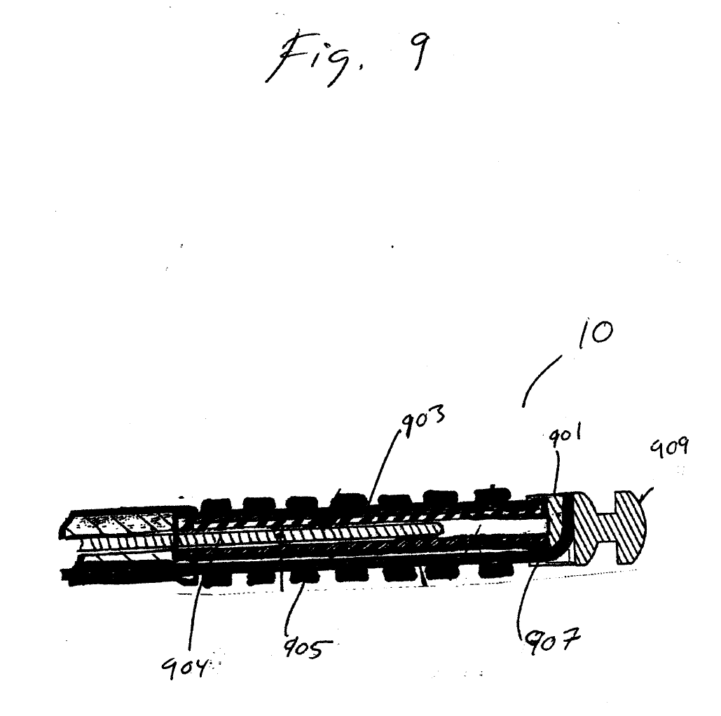

FIGS. 4 a-c show various views of an articulating directional tip assembly consisting of multiple angled interlocking segments.

FIGS. 5 a-d show various views of a preferred embodiment of an interlocking segment.

FIGS. 6 a-b show two perspectives of an articulating tip of an osteotome assembly with a distal cutting tip.

FIGS. 7 a-d show various views of an alternative designed interlocking segment.

FIGS. 8 a-d show various views of a distal cutting tip.

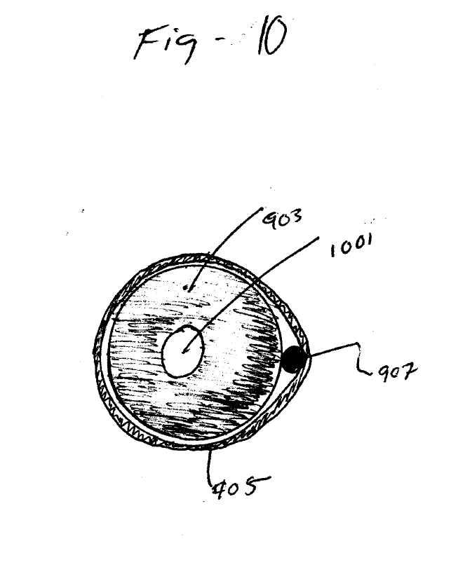

FIG. 9 shows an alterative articulating directional tip assembly design.

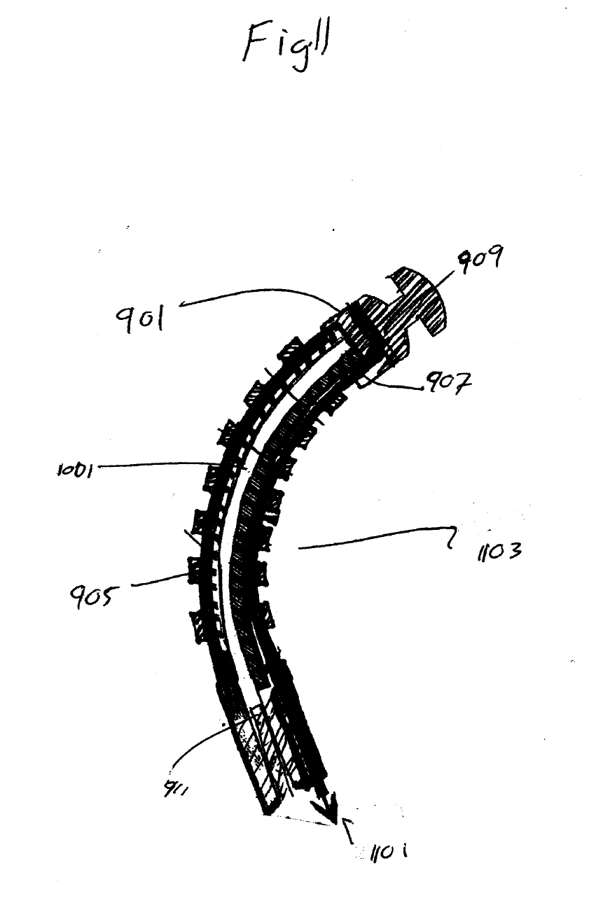

FIG. 10 shows a cutaway of a directional articulating tip assembly shaft.

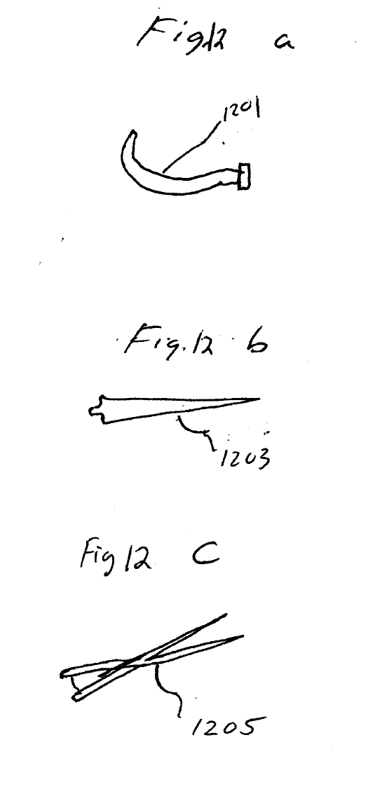

FIG. 11 shows an articulated articulating directional tip assembly.

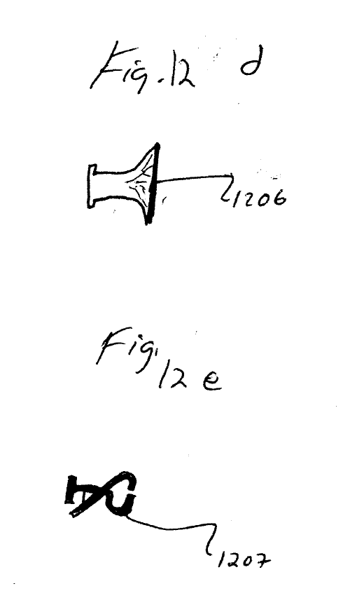

FIG. 12 a-e show various alternative applicators for use with an articulating directional tip assembly.

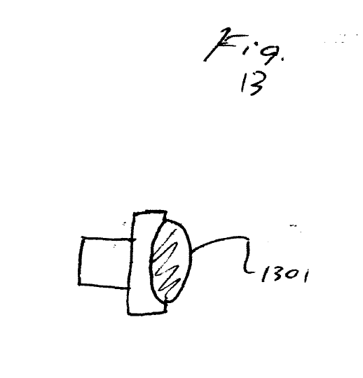

FIG. 13 shows an imaging applicator.

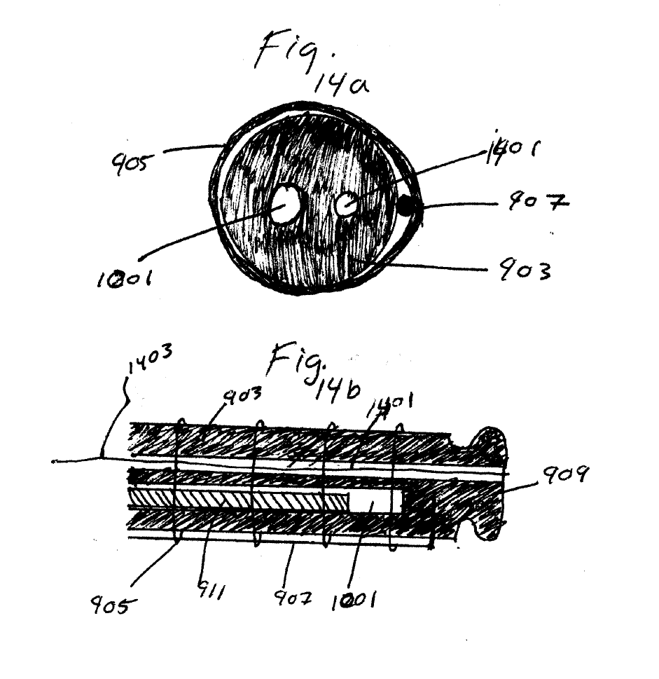

FIG. 14a shows a cutaway of a double lumen shaft.

FIG. 14b shows a side view of a double lumen shaft.

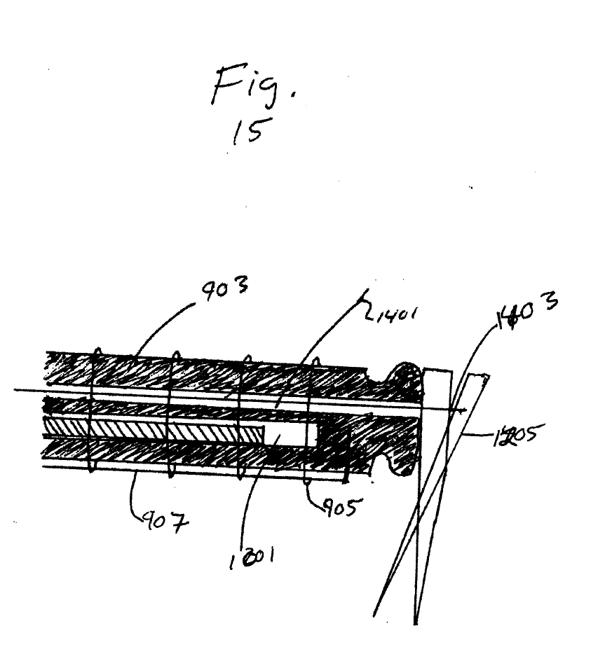

FIG. 15 shows a side view of double lumen shaft with a scissor applicator.

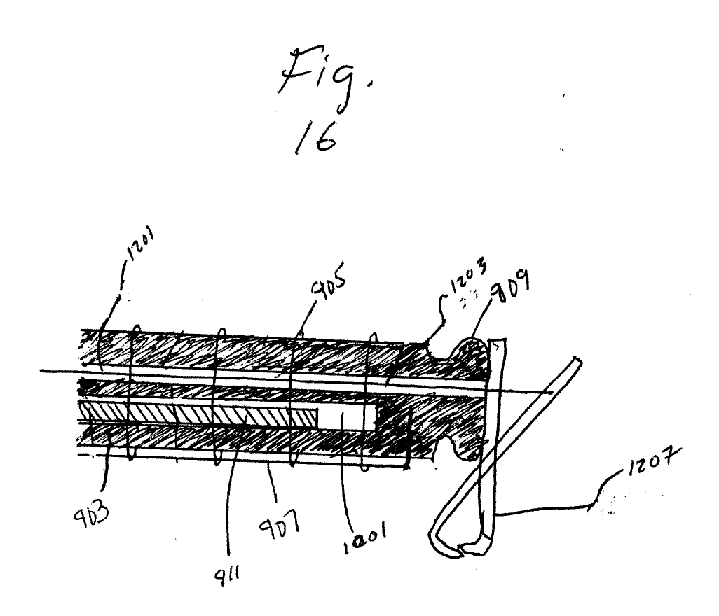

FIG. 16 shows a side view of a double lumen shaft with a bone crusher applicator.

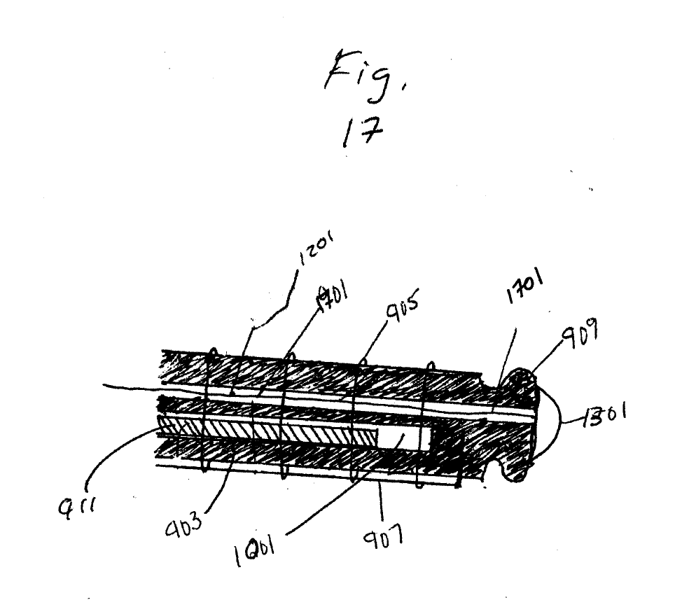

FIG. 17 shows a side view of a double lumen shaft with an imaging applicator.

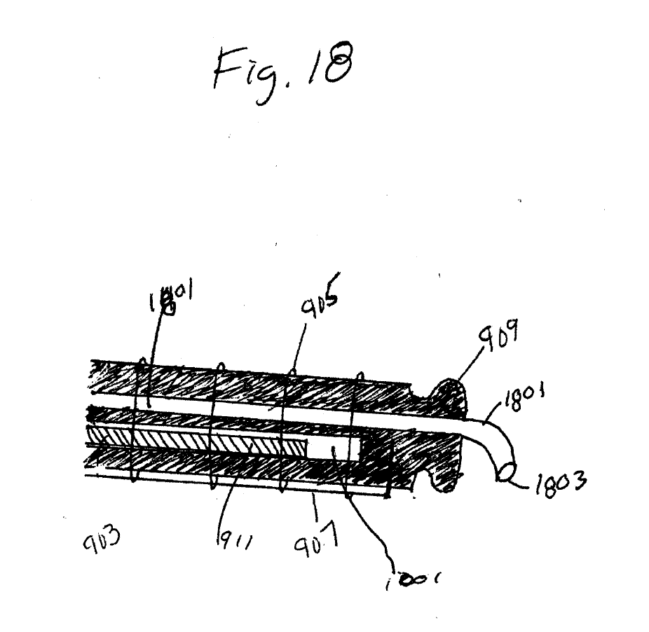

FIG. 18 shows a suctioning lumen with suction applicator.

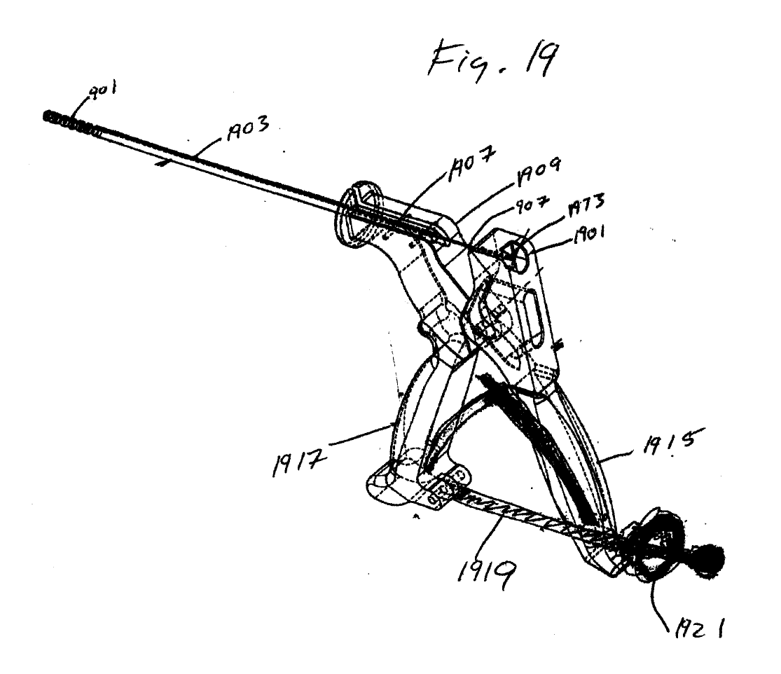

FIG. 19 shows a delivery device with a straight articulating directional tip assembly.

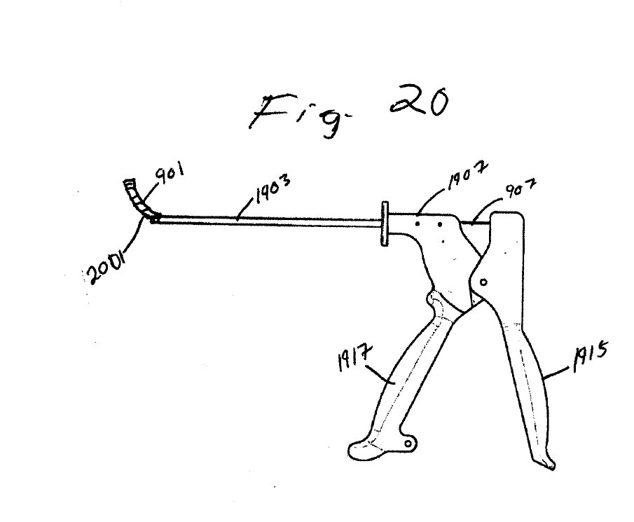

FIG. 20 shows a delivery device with an articulating directional tip assembly.

### DETAILED DESCRIPTION

In the drawings reference numeral 10 generally denotes an exemplary embodiment of an articulating directional tip assembly 201 as shown in FIG. 2.

FIGS. 1 and 2 show an articulating directional tip assembly 201 that consist of two or more interlocking segments 203 that are connected with a flexible linking guidance wire 301. As shown in FIG. 3 the flexible linking guidance wire 301 would connect to the distal tip 205 of the articulating directional tip assembly 201 and connect to or run through a passage within the interlocking segments 203 to the distal end 207 of a delivery cannula 209. A tensioning wire component 305 would also connect to or run through a passage in the interlocking segments then runs through the interior of the delivery cannula 209 to a point where is exits the proximal end of the delivery cannula 211 At the proximal end of the tensioning wire component 305 a tensioning component (not shown) could be connected. That would allow the tensioning wire component 305 to be tensioned proximally through the delivery cannula 209. With the proximal movement of the tensioning wire component 305 the proximal tension of the wire causes the articulation of the directional tip 201 in a desired direction 303. The amount of articulation that can be caused would be correlated to the number of interlocking segments 203 that make up the articulating tip 201 and to the angle 401 of the termination of each interlocking segment based of the design of the segment. In the preferred embodiment the maximum angle would be 15% 403. With this example an articulating tip 201 consisting of six interlocking segments 203 could achieve a 90° angle or articulation 405 with the tensioning of the tensioning wire component 305 by the tensioning component (not shown).

Each interlocking segment 203 would consist of one or more protruding or male components 407 which would interlock with one or more corresponding recessed or female slot 409 on the adjacent segment. FIG. 5 a-d shows a preferred embodiment of an interlocking segment with four protruding or male components 407 and four recessed or female slots 409. Within the interior of the segment in the preferred embodiment there would be one more lumen passages 411 which would allow a flexible linking guidance wire 301 to pass through the interior 501 of the segment from a proximal entry 503 to a distal exit 505. The flexible linking guidance wire 301 and/or the tensioning wire component 305 could be connected anywhere on the interlocking segments however, that would allow for the connecting of the segments and for the articulation of the directional tip.

In FIGS. 4 a-b and 5 a and d three lumen passages are shown that allow for the passage of a flexible linking guidance wire 301, a tensioning wire component 305, and a rigid straightening component, for example, a pin. (not shown.) This rigid straightening component might be utilized to straighten out the articulated directional tip once the tissue altering action within a space is completed to ensure a smooth exit of the assembly from the space. While the preferred embodiment disclosed has four protruding or male components 407 and four recessed or female slots 409, any number of male components and female slots could be utilized that allows the interlocking segments 203 to angle or articulate to the desired angle.

While in the preferred embodiment the angle of the segment termination is 15°, any angle per segment and number of segments utilized could be used that would achieve the maximum desired articulation angle desired. For example, FIG. 6 shows nine interlocking segments 203 that each have a 10° angle 601 on one of the termination end of the respective interlocking segments 203 achieving a 90° articulation upon tensioning of the tensioning wire component 305.

FIGS. 7 a-d show an alternative interlocking segment where the passage 701 that passes through the interior of the segment 203 is rectangular in shape with the width of the passage 701 wider than the height. This would allow for a wide flexible linking guidance wire 301, such as, for example, a band, to pass through proximally to distally and provide a different type of directional control when articulating the articulating tip 201 within a tissue cavity. It also might allow a narrower flexible linking guidance wire 301 that could be moved laterally as well as proximally and distally within the passage 701. FIGS. 7 a, b and d also show an alternative embodiment where the interlocking segment 203 has two protruding or male components 703 and two recessed or female slots 705 rather than four.

FIG. 8 shows a cutting interlocking segment 801 that is located at the distal end 105 of the articulating tip 201 of the articulating directional tip assembly 201. In the preferred embodiment the cutting mechanism 803 is spaced distally 805 from the base of the segment 807 for cutting purposes. Any cutter design could be utilized that would allow for the cutting of material such as for example, body tissue. The cutting interlocking segment 801 would include one or more protruding or male components 407 for interlocking with its proximal interlocking segment 203, it would not include any recess or female slots at its distal end. In one alternative embodiment the osteotome assembly might include a passage through the interior of the delivery cannula 109 and the articulating tip 103 that would exit on the cutting tip as shown in FIG. 8a. This passage might be utilized for providing suction from the cutting tip for the removal of fluids and/or material such as severed body tissue, or for the delivery of materials such as biologic or non-biologic materials or implants. While FIG. 8 shows one example of a cutting mechanism, any mechanism could be utilized with the tip that would accomplish the objective of the user once the articulating tip is delivered into a space such as a tissue cavity and then the articulating tip 203 is rotated or articulated by the tensioning component to its desired placement within the cavity.

FIG. 9 shows an alternative embodiment of an articulating directional tip assembly 901, whereby the device consists of a shaft 903 made of a strong but deformable material such as a semi-rigid braided stainless steel guidance wire 904. Any structure or material might be utilized that is deformable and has sufficient strength to prepare and/or cut non-soft tissue such as bone tissue.

In an alternative embodiment surrounding the center shaft 903 are guidance wire retaining rings 905 that contain a tensioning wire component 907 that runs parallel to the shaft 903 as shown in FIGS. 9-11. In this alternative embodiment, the tensioning wire might be contained in small wire holders attached to the side of the shaft. Any method of connecting the tensioning wire component 907 to the shaft of the articulating directional tip assembly could be utilized, however, that would cause the shaft to bend when the tensioning wire component is tensioned proximally away from the distal tip such as is shown in FIG. 11.

As shown in FIG. 10 in the preferred embodiment, the shaft 903 contains a lumen 1001 for a straightening ram (not shown) that ensures the shaft 903 will only bend as desired when pressure is applied through the tension of the tensioning wire component 907. This straightening ram 911 might be adjustable either outward distally or proximally within the shaft 903 thereby determining the length of movement of the distal end of the shaft 903 depending on the desired result. For example, the portion of the distal end of the shaft 909 might be a ½ inch. In another use, it might be 1½ inch. Any length of shaft could be utilized for bending the tip by sliding the straightening ram 911 distally or proximally within the shaft 903. Once tension is applied on the tensioning wire component 907, the tip 909 is pulled inwardly and directionally 1101 to a desired location as shown in FIG. 11. The tension could be achieved by any mechanism that would pull the tensioning wire component 907 proximally away from the tip 909 causing the bending 1103 shown in FIG. 11.

In one embodiment at the end of the articulating directional tip shaft 909, the tip might be configured for use with a specific application. In an alternative embodiment, the tip 909 might include a connector mechanism to allow for the attachment of a variety of applicators as described below. In one variation, two or more applicators might be attached to the tip 909.

Applicators might include a scoop 1201 as shown in FIG. 12a, a cutter 1203 such as, for example, a knife as shown in FIG. 12b, scissors 1205 as shown in FIG. 12e, or any other tissue separation component. In one variation as shown in FIG. 12d, the applicators might include a non-soft tissue scraper 1206 such as, for example, a bone scraper. In another variation, the applicator might be a bone crusher 1207 as shown in FIG. 12e. Any applicator capable of preparing and/or the removal of non-soft tissue such as bone might be utilized, however. In one variation, the applicators utilized with the directional tip device might include an imaging device such as a camera lens 1301 as shown in FIG. 13. Any imaging or data collecting device might be utilized, however.

The shaft 903 of the device might contain a secondary lumen 1401 as shown in FIG. 14 that might house a tensioning wire component 1403 for manipulating applicators such as, for example, tissue scissors 1205 as shown in FIG. 15 or a bone crusher 1207 as shown in FIG. 16. In that embodiment, the secondary guidance wire 1403 could be tensioned and untensioned allowing for the opening and closing of the scissors 1205. In an alternative embodiment as shown in FIG. 17, the wire contained within the secondary lumen 1401 might be a data wire 1701 allowing data transfer such as, for example, for imaging to be transferred from an imaging lens 1301. Any data transfer mechanism might be utilized.

In another embodiment, the secondary lumen might be a hollow pathway allowing for the suctioning of tissue and/or fluids from a cavity as shown in FIG. 18. In that embodiment, the tip 909 might include a suctioning tip 1801 with an opening 1803 that connects to the secondary lumen 1401 within the shaft 903. A suctioning collector device (not shown) would be connected to the secondary lumen at the proximal end of the shaft. A control device might be included for regulating the suction force as needed. A collection vessel might be utilized to collect the tissue and/or fluid. In alternative embodiment materials such as, for example, biologic or non biologic materials and/or implants could be delivered through a secondary lumen, surrounding The articulating tip or via the tip itself.

FIG. 19 shows an alternative embodiment of a delivery device for an articulating directional tip assembly 1901. The delivery device would consist of a delivery cannula 1903 containing an articulating directional tip assembly 1901. The proximal end of the alternative delivery cannula 1902 is contained in a delivery housing 1909. The proximal end of the tensioning wire component 305 passes through the delivery housing 1909 to a tensioning housing 1911 where it is secured 1913. When tension is desired on the tensioning wire component 305, the user would move the flexible handle 1915 inwardly towards the static handle 1917. This would pull the tensioning wire component 305 distally away from the directional tip 901 causing the tip to articulate 2001 as shown in FIG. 20. In one variation, the squeezable handle 1915 could be locked into position once the desired articulation on the articulating directional tip 901 is achieved. This might be accomplished with a locking mechanism that consist of a guidance wire 1919 connected to the static handle 1917 with a tensioning component 1921 located on the proximal side of the squeezable handle 1915.

It will be appreciated by persons skilled in the art that numerous variations and/or modifications may be made to the described device as specifically shown here without departing limn the spirit or scope of that broader disclosure. The various examples are, therefore, to be considered in all respects as illustrative and not restrictive.

We claim:

1. An articulating directional tip assembly for altering and/or viewing tissue within a tissue cavity, the articulating directional tip assembly comprising:
a directional tip comprised of two or more interlocking segments wherein each segment has one or more male components, and one or more female recesses, with angular terminations at the proximal and/or distal ends of the segment; and
a flexible linking guidance wire component; and
a tensioning wire component;
wherein, the at least two or more ting segments are connected by the flexible linking guidance wire component and are delivered into the tissue cavity, and when in place within the cavity the tensioning wire component is tensioned proximally causing the at least two or more interlocking segments comprising the directional tip to articulate at an angle or arc for altering and/or viewing tissue within the tissue cavity.

2. The assembly of claim 1, wherein the articulating directional tip assembly is delivered through a delivery cannula.

3. The assembly of claim 1, wherein the articulating directional tip assembly's at least two or more interlocking segments each include four male components and four female recesses.

4. The assembly of claim 1, wherein the one or more male components angular terminations are angled between 5° and 90°.

5. The assembly of claim 4, wherein the one or more male components angular terminations are angled 15°.

6. The assembly of claim 1, the guidance wire is contained within a passage within the articulating directional tip assembly's at least two or more interlocking segments.

7. The assembly of claim 6, wherein the guidance wire is a cable.

8. The assembly of claim 6, wherein the guidance wire is a band with a wider width than height and the passage within the segments is rectangular.

9. The assembly of claim 1, wherein the distal tip of the articulating directional tip assembly is a tissue cutter.

10. The assembly of claim 1, wherein the articulating directional tip assembly's at least two or more interlocking segments are connected to a slidable rigid straightening component.

11. The assembly of claim 10, wherein the slidable rigid straightening component is contained within a passage within the articulating directional tip assembly's at least two or more interlocking segments.

12. The assembly of claim 1, wherein the articulating directional tip assembly includes a tissue suction component.

13. The assembly of claim 12, wherein the tissue suction component is contained within a passage within the articulating directional tip assembly's at least two or more interlocking segments.

14. The assembly of claim 1, wherein the articulating directional tip assembly's at least two or more interlocking segments contain three passages, one each for a guidance wire, a rigid straightening component, and a tissue suctioning component.

15. The assembly of claim 1, wherein the distal end of the articulating directional tip assembly includes one of a cutting knife, a scissor, or other tissue separation component.

16. The assembly of claim 1, wherein The assembly of claim 12, wherein the distal end of the articulating directional tip assembly includes one of a scraper, a compactor, a crusher, or a cautery component.

17. The assembly of claim 1, wherein the distal end of articulating directional tip assembly includes one of an imaging component, or a data collecting component.

18. The assembly of claim 17, wherein a transfer wire for the at least one of an imaging component or a data collecting component is contained within a passage within the articulating directional tip assembly's at least two or more interlocking segments.

19. The assembly of claim 17, wherein the imaging device is a camera.
